(19) United States
(12) Patent Application Publication
(10) Pub. No.: US 2007/0154955 A1
(43) Pub. Date: Jul. 5, 2007

(54) Method of diagnosing autoimmune polyglandular syndrome and kits thereof

(75) Inventors: Tzu-Ling Tseng, Chiayi City (TW); Ping-Fu Cheng, Changhua County (TW); Shyh-Shin Chiou, Kaohsiung City (TW)

Correspondence Address: BIRCH STEWART KOLASCH & BIRCH, PO BOX 747, FALLS CHURCH, VA, 22040-0747, US

(73) Assignee: Industrial Technology Research Institute, Hsinchu (TW)

(21) Appl. No.: 11/320,665

(22) Filed: Dec. 30, 2005

(51) Int. Cl. G01N 33/567; G01N 33/53

(52) U.S. Cl. 435/7.2; 435/7.92

(57) ABSTRACT

A method of diagnosing autoimmune polyglandular syndrome (APS) in a subject, and a kit for identifying a subject having autoimmune polyglandular syndrome (APS) are provided.

### BACKGROUND

The invention relates to autoimmune disorders. More particularly, the invention relates to autoantigens specific to autoimmune polyglandular syndrome.

The endocrine system is responsible for the release of hormones into the blood or lymph. Deficiencies in the endocrine system can be caused by infection, infarction, or a tumor destroying all or a large part of the gland. However, the activity of an endocrine organ is most often depressed as a result of an autoimmune reaction that ultimately results in partial or complete destruction of the gland. Autoimmune disease affecting one organ is frequently followed by the impairment of other glands, resulting in multiple endocrine failures.

Autoimmune polyglandular syndromes (APS, also known as polyglandular autoimmune (PGA) syndrome, autoimmune endocrine failure syndrome, polyglandular failure syndromes, autoimmune polyendocrine syndrome, or immunoendocrinopathy syndrome) are constellations of multiple endocrine gland insufficiencies. Essentially, 2 types exist, type I and the more common type II. APS 1 (autoimmune polyglandular syndrome type I), also known as autoimmune polyendocrinopathy-candidiasis-ectodermal dystrophy (APECED) or Whitaker syndrome, is associated with candidiasis, hypoparathyroidism, and adrenal failure. A syndrome with these features was first described in 1946. It is rare disorder with sporadic autosomal recessive inheritance. APS 2 (autoimmune polyglandular syndromes type II) is the most common of the immunoendocrinopathy syndromes. It usually is defined as primary adrenal insufficiency with either autoimmune thyroid disease or type 1 diabetes mellitus occurring in the same individual. Primary hypogonadism, myasthenia gravis, and celiac disease also are commonly observed in this syndrome. The definition of the syndrome depends on the fact that if one of the component disorders is present, an associated disorder occurs more commonly than in the general population. Type III has been described, which occurs in adults and does not involve the adrenal cortex, but it includes 2 of the following: thyroid deficiency, pernicious anemia, insulin-requiring diabetes, vitiligo, and alopecia.

The diagnosis of APS is mainly based on the clinical history of the patient; however, the patient may have severe multi-organ destruction while being diagnosed. A method for the detection of autoimmune polyglandular syndrome is, therefore, still required.

### SUMMARY

Accordingly, an embodiment of the invention provides A method of diagnosing autoimmune polyglandular syndrome (APS) in a subject comprising: (a) obtaining a biological sample from the subject; and (b) determining an amount of at least one autoantibody in the biological sample, wherein the autoantibody specifically binds at least one protein selected from (1) nucleolin; (2) B23 nucleophosmin (280 AA); (3) peptidyl-prolyl cis-trans isomerase B precursor (PPIase); (4) SET protein (HLA-DR associated protein II, I2PP2A); (5) activated RNA polymerase II transcriptional coactivator p15; (6) alpha-fetoprotein precursor (alpha-fetoglobulin or alpha-1-fetoprotein); (7) alpha-tubulin; (8) transformation upregulated nuclear protein (hnRNP K); (9) chain A (nucleoside triphosphate); (10) cytokeratin 8 (279 AA); (11) DNA-binding protein (hnRNP D0); (12) heterogeneous nuclear ribonucleoprotein C isoform b; (13) heterogeneous ribonuclear particle protein A1.beta; (14) MTHSP75; (15) nonhistone chromosomal protein HMG-1; (16) ribosomal protein P2 (60S acidic ribosomal protein P2); (17) ribosomal protein S28 (40S ribosomal protein S28); (18) complete sequence of human pre-mRNA splicing factor SF2p32; and (19) the combination thereof. The presence of the autoantibody in the biological sample indicates that the subject has autoimmune polyglandular syndrome.

Also provided is a kit for identifying a subject having autoimmune polyglandular syndrome (APS). The kit comprises reagents for determining an amount of at least one autoantibody that specifically binds one or more proteins selected from (1) nucleolin; (2) B23 nucleophosmin (280 AA); (3) peptidyl-prolyl cis-trans isomerase B precursor (PPIase); (4) SET protein (HLA-DR associated protein II, I2PP2A); (5) activated RNA polymerase II transcriptional coactivator p15; (6) alpha-fetoprotein precursor (alpha-fetoglobulin or alpha-1-fetoprotein); (7) alpha-tubulin; (8) transformation upregulated nuclear protein (hnRNP K); (9) chain A (nucleoside triphosphate); (10) cytokeratin 8 (279 AA); (11) DNA-binding protein (hnRNP D0); (12) heterogeneous nuclear ribonucleoprotein C isoform b; (13) heterogeneous ribonuclear particle protein A1.beta; (14) MTHSP75; (15) nonhistone chromosomal protein HMG-1; (16) ribosomal protein P2 (60S acidic ribosomal protein P2); (17) ribosomal protein S28 (40S ribosomal protein S28); (18) complete sequence of human pre-mRNA splicing factor SF2p32; and (19) the combination thereof.

### DETAILED DESCRIPTION

A method of diagnosing autoimmune polyglandular syndrome (APS) or a predisposition to developing autoimmune polyglandular syndrome in a subject, and a kit for the detection of autoantigens related to autoimmune polyglandular syndrome (APS) are provided.

Autoimmune polyglandular syndrome (APS), first described as a disease entity by Neufeld, is characterized with multiple endocrine glands insufficiencies (Neufeld M, Maclaren N, Blizzard R. Pediatr Ann 1980; 9:154-162). Essentially, 2 major types of APS exist, APS 1 and the more common APS II which occurs in adulthood mainly in the third or fourth decade (Neufeld M, Maclaren N, Blizzard R. Pediatr Ann 1980; 9:154-162; Jenkins R C, Weetman A P. Thyroid 2002; 12:977-988). APS I, also known as autoimmune polyendocrinopathy-candidiasis-ectodermal dystrophy (APECED) or Whitcker syndrome, usually occurs in children aged 3-5 years or in early adolescence. Three major components of APS I are chronic mucocutaneous candidiasis, chronic hypoparathyroidism, and autoimmune adrenal insufficiency (Finnish-German APECED Consortium. An autoimmune disease, APECED, caused by mutations in a novel gene featuring two PHD-type zinc-finger domains. Nat Genet 1997;17:399-403; Obermayer-Straub P, et al. Clin Rev Allergy Immunol. 2000; 18(2):167-83). At least 2 components have to be present in an individual. Various other manifestations, including hypothyroidism, diabetes, hypogonadism, pernicious anemia, chronic hepatitis, intestinal malabsorption, nail dystrophy and vitiligo may be present as well. Candidiasis usually is the first clinical manifestation, most often presenting in people young than 5 years. Hypoparathyroidism occurs next, usually in people younger than 10 years. Lastly, Addison disease usually occurs in people young than 15 years (Obermayer-Straub P, et al. Clin Rev Allergy Immunol. 2000; 18(2):167-83). APS 1 is a rare disorder that is more frequent in certain isolated populations, such as Finns, Sardinians, and Iranian Jews with sporadic autosomal recessive inheritance (Ahonen P, et al. N Engl J Med 1990; 322:1829-1836; Zlotogora J, et al. J Med Genet 1992; 29:824-826; Rosatelli M C, et al. Hum Genet 1998; 103:428-434). The pathophysiology of APS is based on the presence of a chronic inflammatory infiltrate mainly composed of lymphocytes in the affected organs and on the presence of autoantibodies reacting to target tissue-specific antigens. Development of autoimmunity may be due to shared epitope(s) of an environmental agent and a common antigen present in endocrine tissues (Kamradt T, et al. N Engl J Med 2001; 344:655-664). Furthermore, common germ layer-specific antigens in the endocrine organs derived from the same germ layer have been suggested to serve as targets for the autoimmune responses in APS (Tadmor B, et al. Lancet 1992; 339:975-978). Lack of spontaneous animal models of complete APS may contribute to poor understanding of the pathogenesis of APS. The genetic locus responsible for the disease has been localized to the short arm of chromosome 21 near markers D21s49 and D21s171 on 21q22.3 (Finnish-German APECED Consortium. An autoimmune disease, APECED, caused by mutations in a novel gene featuring two PHD-type zinc-finger domains. Nat Genet 1997; 17:399-403. 10; Aaltonen J, et al. Nature Genet 1994;8: 83-87). The recently identified AIRE (autoimmune regulator) gene represents a candidate gene for APS I (Nagamine K, et al. Nat Genet 1997; 17:393-398; Mittaz L, et al. Biochem Biophys Res Co 1999; 255:483-490; Heino M, et al. Hum Mutat 2001; 18:205-211; and Bjorses P, et al. Am J Hum Genet 2000; 66:378-392). AIRE gene encoding a 58-kDa protein with structural motifs suggestive of a transcription factor featuring a conserved nuclear localization signal, 2 zinc finger (PHD finger) motifs a proline-rich region and four LXXLL or nuclear receptor box motifs (Finnish-German APECED Consortium. An autoimmune disease, APECED, caused by mutations in a novel gene featuring two PHD-type zinc-finger domains. Nat Genet 1997; 17:399-403; Nagamine K, et al. Nat Genet 1997; 17:393-398; Mittaz L, et al. Biochem Biophys Res Co 1999; 255:483-490). Finnish study reported that the mutation R257X in exon 6 of AIRE gene has been shown to be responsible for 82% of cases (Bjorses P, et al. Am J Hum Genet 2000; 66:378-392). A 13-bp deletion in exon 8 was reported to account for 71% of the mutations in APS 1 patients in United Kingdom (Pearce SHS, et al. Am J Hum Genet 1998; 63:1675-1684).

The most frequent disease component in APS 1 is chronic mucocutaneous candidiasis, which was detected in almost all patients at least periodically (Obermayer-Straub P, et al. Clin Rev Allergy Immunol. 2000; 18(2):167-83; Ahonen P, et al. N Engl J Med 1990; 322:1829-1836). The presence of candidiasis is consistent with a T-cell defect (Ahonen P, et al. N Engl J Med 1990; 322:1829-1836; Neufeld M, et al. Medicine (Baltimore) 1981; 60:355-362; Betterle C, et al. J Clin Endocrinol Metab 1998; 83:1049-1055). Hypoparathyroidism is the most frequent endocrine disease component in APS 1 and affects about 80-90% of all patients (Neufeld M, et al. Pediatr Ann 1980; 9:154-162; Obermayer-Straub P, et al. Clin Rev Allergy Immunol. 2000; 18(2):167-83; Ahonen P, et al. N Engl J Med 1990; 322:1829-1836). Autoantibodies against parathyroid epithelia, endothelia and Ca2+-sensing receptor have been reported in 10-40% of patients with hypoparathyroidism (Miettinen A, et al. Pediatr Res 1982; 16:889; Brandi M-L, et al. Proc Natl Acad Sci USA 1986; 83:8366-8369; Perniola R, et al. Eur J Endocrinol 2000; 143:497-503; Fattorossi A, et al. Proc Natl Acad Sci USA 1988; 85:4015-4019; and Li Y, et al. J Clin Invest 1996; 97:910-914). The pathological significance of these antibodies is not clear. Adrenal failure in APS 1 can be diagnosed from age 4-41 and is associated with the presence of steroidal cell autoantibodies. Autoantibodies P450 cytochrome enzymes SCC, 17-OH, and 21-OH have been associated with Addison's disease in APS 1 (Heino M, et al. Biochem Biophys Res Commun 1999; 257:821-825; Su D, et al. Dev Biol 2001; 236:316-329; and Xu P-X, et al. Development 2002; 129:33-3034). Chronic autoimmune hepatitis is associated with liver and kidney microsomal antibodies, reacting with cytochrome P450 1A2 and 2A6 antigens (Michele T M, et al. Postgrad Med J 1994; 70:128-131; Clemente M G, et al. J Clin Endocrinol Metab 1997; 82:1353-1361; and Clemente M G, et al. Gastroenterology 1998; 114:324-328).

Autoantigens Associated with APS

Candidate autoantigens associated with particular autoimmune disorders may be identified by a variety of methods, including, for example, the method described in U.S. Patent Publication No. US-2005/0124076-A1, published on Jun. 9, 2005, which is incorporated herein by reference. According to this method, serum antibodies are purified from serum samples obtained from healthy individuals and from individuals afflicted with an autoimmune disorder of interest, such as APS. The purified immunoglobulins are covalently attached to a chromatographic medium and used to make an affinity column. A protein sample isolated from a subject having APS, for example, taken from an organ, tissue, or cell type involved in the etiology or progression of the disease, is then analyzed.

For example, to identify candidate autoantigens associated with APS, first, the protein sample would be passed over the column containing immunoglobulins isolated from a healthy individual. The unbound proteins are then passed over the column containing immunoglobulins isolated from a patient suffering from APS. All proteins bound to the second column are candidate autoantigens that are then eluted from the column and used in further analysis, such as, for example, mass spectrometry, to identify each isolated protein.

The autoantigens were displaced from the patient antibody packing column and subjected to mass spectrometry analysis. The obtained mass spectra were compared with the database to identify the autoantigens. With this method, eighteen autoantigens were identified, including nucleolin; B23 nucleophosmin (280 AA); peptidyl-prolyl cis-trans isomerase B precursor (PPIase); SET protein (HLA-DR associated protein II) (I2PP2A); activated RNA polymerase II transcriptional coactivator p15; alpha-fetoprotein precursor, alpha-fetoglobulin, alpha-1-fetoprotein; alpha-tubulin; transformation upregulated nuclear protein (hnRNP K); chain A, nucleoside triphosphate; cytokeratin 8 (279 AA); DNA-binding protein (hnRNP D0); heterogeneous nuclear ribonucleoprotein C isoform b; heterogeneous ribonuclear particle protein A1.beta; MTHSP75; nonhistone chromosomal protein HMG-1; ribosomal protein P2, 60S acidic ribosomal protein P2; ribosomal protein S28, 40S ribosomal protein S28; human pre-mRNA splicing factor SF2p32, complete sequence.

Accordingly, one embodiment of the invention provides a method of diagnosing autoimmune polyglandular syndrome (APS) in a subject. The method comprises (a) obtaining a biological sample from the subject; and (b) determining an amount of at least one autoantibody in the biological sample. The autoantibody specifically binds at least one protein selected from (1) nucleolin; (2) B23 nucleophosmin (280 AA); (3) peptidyl-prolyl cis-trans isomerase B precursor (PPIase); (4) SET protein (HLA-DR associated protein II, I2PP2A); (5) activated RNA polymerase II transcriptional coactivator p15; (6) alpha-fetoprotein precursor (alpha-fetoglobulin or alpha-1-fetoprotein); (7) alpha-tubulin; (8) transformation upregulated nuclear protein (hnRNP K); (9) chain A (nucleoside triphosphate); (10) cytokeratin 8 (279 AA); (11) DNA-binding protein (hnRNP D0); (12) heterogeneous nuclear ribonucleoprotein C isoform b; (13) heterogeneous ribonuclear particle protein A1.beta; (14) MTHSP75; (15) nonhistone chromosomal protein HMG-1; (16) ribosomal protein P2 (60S acidic ribosomal protein P2); (17) ribosomal protein S28 (40S ribosomal protein S28); (18) complete sequence of human pre-mRNA splicing factor SF2p32; and (19) the combination thereof. The proteins (1) to (18) comprise the sequences of SEQ ID NO: 1 to SEQ ID NO: 18 respectively. In addition, the presence of the autoantibody in the biological sample indicates that the subject has autoimmune polyglandular syndrome.

In one embodiment of the method of diagnosing autoimmune polyglandular syndrome (APS) in a subject, the determination of the presence of the autoantigens is by immunoassay. The immunoassay can be enzyme immunoassay (EIA), radioimmunoassay (RIA), fluorescent immunoassay (FIA), or enzyme-linked immunosorbent assay (ELISA).

In another embodiment of the method of diagnosing autoimmune polyglandular syndrome (APS) in a subject, the biological sample can be blood, serum, or body fluid of the subject.

Also provided is a kit for identifying a subject having autoimmune polyglandular syndrome (APS). The kit comprises reagents for determining an amount of at least one autoantibody that specifically binds one or more proteins selected from (1) nucleolin; (2) B23 nucleophosmin (280 AA); (3) peptidyl-prolyl cis-trans isomerase B precursor (PPIase); (4) SET protein (HLA-DR associated protein II, I2PP2A); (5) activated RNA polymerase II transcriptional coactivator p15; (6) alpha-fetoprotein precursor (alpha-fetoglobulin or alpha-1-fetoprotein); (7) alpha-tubulin; (8) transformation upregulated nuclear protein (hnRNP K); (9) chain A (nucleoside triphosphate); (10) cytokeratin 8 (279 AA); (11) DNA-binding protein (hnRNP D0); (12) heterogeneous nuclear ribonucleoprotein C isoform b; (13) heterogeneous ribonuclear particle protein A1.beta; (14) MTHSP75; (15) nonhistone chromosomal protein HMG-1; (16) ribosomal protein P2 (60S acidic ribosomal protein P2); (17) ribosomal protein S28 (40S ribosomal protein S28); (18) complete sequence of human pre-mRNA splicing factor SF2p32; and (19) the combination thereof in a biological sample. The proteins (1) to (18) comprise SEQ ID NO: 1 to SEQ ID NO: 18 respectively.

In one embodiment of the kit for identifying a subject having APS, the detection of the autoantigen is by immunoassay. The immunoassay can be enzyme immunoassay (EIA), radioimmunoassay (RIA), fluorescent immunoassay (FIA), or enzyme-linked immunosorbent assay (ELISA).

In another embodiment of the kit for identifying a subject having APS, the kit is a microarray.

Practical examples are described herein.

### EXAMPLES

1. Patient History:

The 15-year-6-month old boy was born with a birth body weight 3780 gm. No obvious family history of endocrine diseases was noted. He was admitted in a teaching hospital due to severe mucocutaneous candidiasis and fever at 2 year of age and had periodically recurrent chronic mucocutaneous candidiasis during his childhood. The patient was admitted in Kaohsiung Medical University Hospital, Taiwan, at 5 year of age with tetanic carpopedal spasm and generalized tonic-clonic seizure secondary to severe hypocalcemia (serum ionized calcium levels were 1.9 mg %; normal reference range 4.0-5.5 mg %). Hypoparathyroidism was diagnosed based on undetectable serum parathyroid hormone levels (less than 1 pg/ml; normal laboratory range 10-65 pg/ml) and hyperphophatemia (inorganic phosphorus 9.7 mg/dl; normal laboratory range 2.7-4.7 mg/dl). The clinical symptoms were controlled after treatment with hydroxyvitamine D3, intravenous and oral calcium replacement. Recurrent episodes of tetany and seizure occurred and were treated with additional calcium supplement. Before teenage period, he suffered from episodes of pneumonia, submandibular cellulites and herpes infection and once was admitted in the intensive care unit due to severe illness. T cell immunodeficiency was diagnosed because of low active T lymphocyte percentage (5.9%; normal laboratory range 26.0-39.2%) in his peripheral blood and abnormal candida delayed skin test with an induration less than 1 cm after 48 and 72 hours of inoculation. Digeorge syndrome was excluded because the thymus was normal in the chest X-ray film and no anomalies of great vessels, esophagus or face structures were found. Chronic active hepatitis was diagnosed by liver biopsy while the patient presented with persistent jaundice, hepatomegaly and impaired liver function. After exclusion of viral infection and other possible causes, autoimmune hepatitis was diagnosed according to clinical manifestation, positive antinuclear antibodies (titer 1:640) and low complement level (C4: 13.2 mg/dl; normal laboratory range 17.2-32.8 mg/dl) at 12 year of age. Megaloblastic anemia (MCV 114.5 fl) was subsequently found. He also suffers from gall bladder stone and nephrocalcinosis complicating with distal tubular acidosis, hypomagnesemia and urinary acidification defect because of possible inappropriate calcium supplement. At 17 year of age, the patient was found adrenocortical failure with glucocorticoid (cortisol 2.9 mg/dl at 0900 h; normal laboratory range 5-23 mg/dl) and mineralcorticoid deficiency (aldosterone in upright position <2.5 ng/dl; normal laboratory range 7-30 ng/dl) featuring lethargy, hyponatremia (sodium 123 mmol/l; normal laboratory range 135-145 mmol/l), and hypochloremia (chloride 89 mmol/l; normal laboratory range 98-106 mmol/l).

Physical examination also shows vitiligo on his face, nail dystrophy and onychomycosis. Chromosome study was normal. No thyroid disease, diabetes or hypogonadism was found. Autoimmune polyglandular syndrome type I (APS I) was therefore diagnosed.

2. Purification of Antibodies (IgG) from Sera:

Serum samples were obtained from a healthy adult (Show Chwan Health Care System) and the patient as described above and subjected to affinity column purification. Samples were diluted with binding buffer (20 mM PBS, pH 7.0) at a ratio of 1:10 and filtrated trough a 0.45 μm filter (MILLEX HV 0.45 μm filter, Millipore). The filtration step was to prevent the possible blockage of the column in the subsequent steps. Protein G affinity column (HiTrap™ Protein G HP Columns, Amersham Biosciences) was applied for the purification. The column was first rinsed with 10 CV (column volume) of binding buffer at a flow rate of 1 ml/min, the samples were then applied at a flow rate of 0.2 ml/min, and the column was washed with 5˜10 CV of binding buffer at a flow rate of 1 ml/min. Substances which do not bind to the column were removed by the washing step. Elution was performed with 2˜5 CV of elution buffer (0.1 M Glycine-HCl, pH 2.7) at a flow rate of 1 ml/min. The eluted antibodies were collected in collection tubes containing 60˜200 μl of 1M Tris-HCl, pH 9.0. The collected antibodies were displaced with coupling buffer (0.2 M NaHCO₃, 0.5M NaCl, pH 8.3) for the subsequent steps. The collected antibodies obtained from the APS patients contain autoantibodies.

3. Preparation of Column Containing the Collected Antibodies

Columns containing normal antibodies and autoantibodies were prepared with the collected antibodies obtained from sera of the healthy adult and the APS patient, respectively, as described below. Few amounts of acidification solution (1 mM HCl, ice bathed) were applied to a NHS-activated column for preventing bubble formation prior to the preparation. Isopropanol contained in the column was rinsed out by 2 CV of acidification solution for three times. One CV of the coupling buffer containing the collected antibodies (normal antibodies or autoantibodies) in a concentration of 0.5˜10 mg/ml were then injected into the column by a syringe connected to the upper end of the column, and the column was sealed immediately for 15-30 min reaction under 25° C. or for 4-hour reaction under 4° C. The antibodies could be immobilized to the column through chemical bonding. After the reaction, elution was performed with 2 CV of blocking buffer (0.5M ethanolamine, 0.5M NaCl, pH 8.3) for three times, and the column was rinsed with 2 CV of washing buffer (0.1M acetate, 0.5M NaCl, pH 4) for three times. The secondary elution was performed with 3 CV of blocking buffer. The column was then left for 15˜30 min reaction to block and inactivate the functional groups in the column which did not bind to the applied antibodies. After the blocking reaction, the column was rinsed with 2 CV of washing buffer for three times, followed by eluted with 2 CV of blocking buffer for three times, ensuring that all functional groups which do not bind to the antibodies are blocked. The column was rinsed again with 2 CV of washing buffer for three times. Finally, the column was eluted again with 2˜5 CV of pH neutral buffer, and normal antibody and autoantibody packing column were obtained for the subsequent steps.

4. Purification of Autoantigens from Cell Extract Obtained from APS Patient

Cell extract was obtained from HepG2 C3A cells. Removal of the culture medium was by ice-bathed Tris saline solution (50 mM Tris pH 7.5, 150 mM NaCl, 1.5 mM PMSF, phosphatase inhibitors) twice. One ml of Triton extraction solution (15 mM Tris pH 7.5, 120 mM NaCl, 25 mM KCl, 2 mM EGTA, 0.1 mM DTT, 0.5% Triton X-100, 10 μg/ml leupeptin, 0.5 mM PMSF, and phosphatase inhibitors) was added into the cell extract for 30 minutes under 4° C. in order to decompose the cells for protein releasing. Solid and insoluble cell structure was removed by centrifugation using a tabletop centrifuge at 14,000 rpm under 4° C. for 15 minutes. The supernatant was collected for immunoaffinity chromatography.

Cell extract was diluted with binding buffer (20 mM PBS, pH 7.0) at a ratio of 1:10 and filtrated trough a 0.45 μm filter (MILLEX HV 0.45 μm filter, Millipore). The filtration step was to prevent the possible blockage of the column in the subsequent steps. The normal antibody and autoantibody packing columns were first rinsed with 10 CV (column volume) of binding buffer at a flow rate of 1 ml/min. The filtrated cell extract was then applied to the normal antibody packing column at a flow rate of 0.2 ml/min, and the normal antibody packing column was washed with 5˜10 CV of binding buffer at a flow rate of 1 ml/min. Antigens in the cell extract that were identified and captured by the normal antibodies would be retained in the column, and the resulting cell extract was free of non-specific antigens. The resulting cell extract was then subject to the autoantigen packing column. The column was washed with 5˜10 CV of binding buffer at a flow rate of 1 ml/min. Autoantigens presented in the cell extract would be captured by the autoantibodies of the column. The captured autoantigens were eluted with 2˜5 CV of elution buffer (0.1 M Glycine-HCl, pH 2.7) and collected in collection tubes containing 60-200 μl of 1M Tris-HCl, pH 9.0.

4. Identification of Autoantigens from Cell Extracts

The collected autoantigens were subjected to trypsin digestion, followed by mass spectrometric analysis. For trypsin digestion, the collected autoantigens were solublized in 100 μl of 6M urea, and 5 μl of 200 mM DTT was added to the solution. After vortex, the solution was placed at 37° C. for 1 hour. Twenty μl of 200 mM Iodoacetamide was added, and the solution was mixed and reacted in the dark for 1 hour. Twenty μl of 200 mM DTT was again added, and the solution was centrifuged under 12000 g by Microcon MW=3000 Da centrifugal filter (Millipore) at 4° C. for 40 min. Supernatant was collected and added with 50 to 100 μl of ammonium bicarbonate, and the solution was centrifuged under 14000 g at 4° C. for 40 min. The step was repeated three times. Trypsinization was performed with trypsin to the sample of 1:50 under water bath at 37° C. for 20 hours. Reaction was terminated by 5˜10 μl of formic acid. The resulting solution was concentrated with a vacuum and stored at −20 C.

For mass spectrometry, the obtained peptides were applied to NanoLC system for separation and desalting. The NanoLC system was NanoHPLC system (LC packings, Netherlands) with C18 column (75 μm I.D.×150 mmL, 3 μm particles). Elution was performed with a linear gradient from 5% solvent B (0.1% w/v formic acid/H₂O/80% v/v acetonitrile) to 50% solvent B over 45 min and then with 95% solvent B for 30 min balance. The obtained peptides were dissolved in solvent A (solvent A (0.1% w/v formic acid/H₂O/2% v/v acetonitrile) and loaded into C18 column. Some peptides might bind to the column tially and would be eluted by the gradually increased vent B.

The peptides were then applied to QSTAR mass analyzer (ESI-Q/TOF MS, Applied life Science). Peptides with specific m/z value were subjected to MS/MS and MS/MS spectra these peptides were obtained.

The obtained peptide MS/MS spectra were compared with NCBInr human database using Mascot Search Software.

Eighteen autoantigens were screened out as listed in Table 1.

TABLE 1
Autoantigens for APS

| | Matched | | | | SEQ |
|---|---|---|---|---|---|
| | peptide | | | | ID |
| No. | number | Score* | Protein | MW (kD) | NOs |
| 1 | 8 | 428 | Nucleolin | 76 | 1 |
| 2 | 4 | 142 | B23 nucleophosmin | 32 | 2 |
| | | | (280 AA) | | |
| 3 | 4 | 186 | Peptidyl-prolyl | 22 | 3 |
| | | | cis-trans isomerase | | |
| | | | B precursor (PPIase) | | |
| 4 | 3 | 67 | SET protein (HLA-DR | 33 | 4 |
| | | | associated protein | | |
| | | | II) (I2PP2A) | | |
| 5 | 2 | 81 | Activated RNA | 14 | 5 |
| | | | polymerase II | | |
| | | | transcriptional | | |
| | | | coactivator p15 | | |
| 6 | 2 | 75 | alpha-fetoprotein | 68 | 6 |
| | | | precursor; alpha- | | |
| | | | fetoglobulin; alpha-1- | | |
| | | | fetoprotein | | |
| 7 | 2 | 85 | alpha-tubulin | 50 | 7 |
| 8 | 2 | 91 | Transformation | 51 | 8 |
| | | | upregulated nuclear | | |
| | | | protein (hnRNP K) | | |
| 9 | 2 | 34 | Chain A, Nucleoside | 42 | 9 |
| | | | Triphosphate | | |
| 10 | 1 | 34 | Cytokeratin 8 | 53 | 10 |
| | | | (279 AA) | | |
| 11 | 1 | 33 | DNA-binding protein | 38 | 11 |
| | | | (hnRNP D0) | | |
| 12 | 1 | 40 | Heterogeneous nuclear | 33 | 12 |
| | | | ribonucleoprotein C | | |
| | | | isoform b | | |
| 13 | 1 | 61 | Heterogeneous | 39 | 13 |
| | | | ribonuclear particle | | |
| | | | protein A1.beta | | |
| 14 | 1 | 46 | MTHSP75 | 73 | 14 |
| 15 | 1 | 36 | Nonhistone chromosomal | 25 | 15 |
| | | | protein HMG-1 | | |
| 16 | 1 | 46 | ribosomal protein P2; | 11 | 16 |
| | | | 60S acidic ribosomal | | |
| | | | protein P2 | | |
| 17 | 1 | 66 | Ribosomal protein S28; | 8 | 17 |
| | | | 40S ribosomal | | |
| | | | protein S28 | | |
| 18 | 1 | 36 | [Human pre-mRNA | 31 | 18 |
| | | | splicing factor SF2p32, | | |
| | | | complete sequence.] | | |

*Score was determined by a software (Matrix Science Mascot ™ v1.8). The score over 33 indicates 95% confidential interval, i.e. p < 0.05.

5. Western Blotting Analysis of Autoantigens for APS

Western blotting analysis was also applied to further confirm the autoantigens for APS. Western blot analysis was as described below. SDS-PAGE was carried out using a 10% polyacrylamide gel. Each lane was loaded with 20 ug cell extract protein. After SDS-PAGE, proteins were transferred to a PVDF membrane. The membrane was blocked with 5% powdered nonfat milk in PBST (10 mM Tris-HCl pH 7.4, 150 mM NaCl, 0.05% Tween 20) for 1 hour and then incubated in 5% powdered non-fat milk-PBST containing APS patient sera at a dilution of 1:1000 for 1 hour at room temperature. Next, the membrane was washed and incubated with the secondary antibody, anti-human IgG-horseradish peroxidase (Biosource International, Camarillo, Calif., USA), at 1:7,500 in PBST/5% powdered non-fat milk for 1 hour at room temperature. Bound oxidase was detected with ECL blotting substrate (pharmacia Biotech) according to the manufacturer's instructions. The results showed that APS autoantigens could be detected by the APS patient's serum. Among these APS autoantigens listed in table 1, nucleolin; alpha-fetoprotein precursor, alpha-fetoglubulin, alpha-1-fetoprotein; transformation upregulated nuclear protein (hnRNP K); alpha-tubulin; chain A, nucleoside triphosphate; B23 nucleophosmin (280 AA); Peptidyl-prolyl cis-trans isomerase B precursor (PPIase); Activated RNA polymerase II transcriptional coactivator p15 were of more significance.

While the invention has been described by way of example and in terms of preferred embodiment, it is to be understood that the invention is not limited thereto.

What is claimed is:

1. A method of diagnosing autoimmune polyglandular syndrome (APS) in a subject comprising:
(a) obtaining a biological sample from the subject; and
(b) determining an amount of at least one autoantibody in the biological sample, wherein the autoantibody specifically binds at least one protein selected from:
(1) nucleolin;
(2) B23 nucleophosmin (280 AA);
(3) peptidyl-prolyl cis-trans isomerase B precursor (PPIase);
(4) SET protein (HLA-DR associated protein II, I2PP2A);
(5) activated RNA polymerase II transcriptional coactivator p15;
(6) alpha-fetoprotein precursor (alpha-fetoglobulin or alpha-1-fetoprotein);
(7) alpha-tubulin;
(8) transformation upregulated nuclear protein (hnRNP K);
(9) chain A (nucleoside triphosphate);
(10) cytokeratin 8 (279 AA);
(11) DNA-binding protein (hnRNP D0);
(12) heterogeneous nuclear ribonucleoprotein C isoform b;
(13) heterogeneous ribonuclear particle protein A1.beta;
(14) MTHSP75;
(15) nonhistone chromosomal protein HMG-1;
(16) ribosomal protein P2 (60S acidic ribosomal protein P2);
(17) ribosomal protein S28 (40S ribosomal protein S28);
(18) complete sequence of human pre-mRNA splicing factor SF2p32; and
(19) the combination thereof;
wherein the presence of the autoantibody in the biological sample indicates that the subject has autoimmune polyglandular syndrome.

2. The method as claimed in claim 1, wherein the determination of the presence of the autoantigens is by immunoassay.

3. The method as claimed in claim 2, wherein the immunoassay is enzyme immunoassay (EIA).

4. The method as claimed in claim 2, wherein the immunoassay is radioimmunoassay (RIA).

5. The method as claimed in claim 2, wherein the immunoassay is fluorescent immunoassay (FIA).

6. The method as claimed in claim 2, wherein the immunoassay is enzyme-linked immunosorbent assay (ELISA).

7. The method as claimed in claim 1, wherein the biological sample is blood, serum, or body fluid of the subject.

8. The method as claimed in claim 1, wherein the proteins (1) to (18) comprise the sequences of SEQ ID NO: 1 to SEQ ID NO: 18 respectively.

9. A kit for identifying a subject having autoimmune polyglandular syndrome (APS) comprising reagents for determining an amount of at least one autoantibody that specifically binds one or more proteins selected from:
(1) nucleolin;
(2) B23 nucleophosmin (280 AA);
(3) peptidyl-prolyl cis-trans isomerase B precursor (PPIase);
(4) SET protein (HLA-DR associated protein II, I2PP2A);
(5) activated RNA polymerase II transcriptional coactivator p15;
(6) alpha-fetoprotein precursor (alpha-fetoglobulin or alpha-1-fetoprotein;
(7) alpha-tubulin;
(8) transformation upregulated nuclear protein (hnRNP K);
(9) chain A, nucleoside triphosphate;
(10) cytokeratin 8 (279 AA);
(11) DNA-binding protein (hnRNP D0);
(12) heterogeneous nuclear ribonucleoprotein C isoform b;
(13) heterogeneous ribonuclear particle protein A1.beta;
(14) MTHSP75;
(15) nonhistone chromosomal protein HMG-1;
(16) ribosomal protein P2 (60S acidic ribosomal protein P2);
(17) ribosomal protein S28 (40S ribosomal protein S28);
(18) complete sequence of human pre-mRNA splicing factor SF2p32; and
(19) the combination thereof in a biological sample.

10. The kit as claimed in claim 9, wherein the detection is by immunoassay.

11. The kit as claimed in claim 10, wherein the immunoassay is enzyme immunoassay (EIA).

12. The kit as claimed in claim 10, wherein the immunoassay is radioimmunoassay (RIA).

13. The kit as claimed in claim 10, wherein the immunoassay is fluorescent immunoassay (FIA).

14. The kit as claimed in claim 10, wherein the immunoassay is enzyme-linked immunosorbent assay (ELISA).

15. The kit as claimed in claim 10, wherein the kit is a microarray.

16. The kit as claimed in claim 9, wherein the proteins (1) to (18) comprise the sequences of SEQ ID NO: 1 to SEQ ID NO: 18 respectively.


SEQUENCE LISTING (18 sequences)

SEQ ID NO: 1 - protein, 710 aa, Homo sapiens
  CHAIN (1)..(710): nucleolin <s300> NCBI/NP_005372.2  GI55956788 2005-12-11 (1)..(710)
MVKLAKAGKNQGDPKKMAPPPKEVEEDSEDEEMSEDEEDDSSGEEVVIPQKKGKKAAATSAKKVVVSPTKKVAVATPAKKAAVTPGKKAAATPAKKTVTPAKAVTTPGKKGATPGKALVATPGKKGAAIPAKGAKNGKNAKKEDSDEEEDDDSEEDEEDDEDEDEDEDEIEPAAMKAAAAAPASEDEDDEDDEDDEDDDDDEEDDSEEEAMETTPAKGKKAAKVVPVKAKNVAEDEDEEEDDEDEDDDDDEDDEDDDDEDDEEEEEEEEEEPVKEAPGKRKKEMAKQKAAPEAKKQKVEGTEPTTAFNLFVGNLNFNKSAPELKTGISDVFAKNDLAVVDVRIGMTRKFGYVDFESAEDLEKALELTGLKVFGNEIKLEKPKGKDSKKERDARTLLAKNLPYKVTQDELKEVFEDAAEIRLVSKDGKSKGIAYIEFKTEADAEKTFEEKQGTEIDGRSISLYYTGEKGQNQDYRGGKNSTWSGESKTLVLSNLSYSATEETLQEVFEKATFIKVPQNQNGKSKGYAFIEFASFEDAKEALNSCNKREIEGRAIRLELQGPRGSPNARSQPSKTLFVKGLSEDTTEETLKESFDGSVRARIVTDRETGSSKGFGFVDFNSEEDAKAAKEAMEDGEIDGNKVTLDWAKPKGEGGFGGRGGGRGGFGGRGGGRGGRGGFGGRGRGGFGGRGGFRGGRGGGGDHKPQGKKTKFE

SEQ ID NO: 2 - protein, 280 aa, Homo sapiens
  CHAIN (1)..(280): B23 nucleophosmin <s300> NCBI/CAA34809.1  GI825671 1995-07-19 (1)..(280)
QNYLFGCELKADKDYHFKVDNDENEHQLSLRTVSLGAGAKDELHIVEAEAMNYEGSPIKVTLATLKMSVQPTVSLGGFEITPPVVLRLKCGSGPVHISGQHLVAVEEDAESEDEEEEDVKLLSISGKRSAPGGGSKVPQKKVKLAADEDDDDDDEEDDDEDDDDDDFDDEEAEEKAPVKKSIRDTPAKNAQKSNQNGKDSKPSSTPRSKGQESFKKQEKTPKTPKGPSSVEDIKAKMQASIEKGGSLPKVEAKFINYVKNCFRMTDQEAIQDLWQWRKSL

SEQ ID NO: 3 - protein, 216 aa, Homo sapiens
  CHAIN (1)..(216): peptidylprolyl isomerase B precursor <s300> NCBI/NP_000933.1  GI475895020051106 2005-11-06 (1)..(216)
MLRLSERNMKVLLAAALIAGSVFFLLLPGPSAADEKKKGPKVTVKVYFDLRIGDEDVGRVIFGLFGKTVPKTVDNFVALATGEKGFGYKNSKFHRVIKDFMIQGGDFTRGDGTGGKSIYGERFPDENFKLKHYGPGWVSMANAGKDTNGSQFFITTVKTAWLDGKHVVFGKVLEGMEVVRKVESTKTDSRDKPLKDVIIADCGKIEVEKPFAIAKE

SEQ ID NO: 4 - protein, 290 aa, Homo sapiens
  CHAIN (1)..(290): SET protein <s300> NCBI/Q01105  GI46397790 2006-01-24 (1)..(290)
MAPKRQSPLPPQKKKPRPPPALGPEETSASAGLPKKGEKEQQEAIEHIDEVQNEIDRLNEQASEEILKVEQKYNKLRQPFFQKRSELIAKIPNFWVTTFVNHPQVSALLGEEDEEALHYLTRVEVTEFEDIKSGYRIDFYFDENPYFENKVLSKEFHLNESGDPSSKSTEIKWKSGKDLTKRSSQTQNKASRKRQHEEPESFFTWFTDHSDAGADELGEVIKDDIWPNPLQYYLVPDMDDEEGEGEEDDDDDEEEEGLEDIDEEGDEDEGEEDEDDDEGEEGEEDEGEDD

SEQ ID NO: 5 - protein, 127 aa, Homo sapiens
  CHAIN (1)..(127): activated RNA polymerase II transcriptional coactivator p15 <s300> NCBI/P53999  GI1709514 2006-01-24 (1)..(127)
MPKSKELVSSSSSGSDSDSEVDKKLKRKKQVAPEKPVKKQKTGETSRALSSSKQSSSSRDDNMFQIGKMRYVSVRDFKGKVLIDIREYWMDPEGEMKPGRKGISLNPEQWSQLKEQISDIDDAVRKL

SEQ ID NO: 6 - protein, 609 aa, Homo sapiens
  CHAIN (1)..(609): alpha-fetoprotein precursor, alpha-fetoglobulin, alpha-1-fetoprotein <s300> NCBI/P02771  GI120042 2006-01-24 (1)..(609)
MKWVESIFLIFLLNFTESRTLHRNEYGIASILDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTPASIPLFQVPEPVTSCEAYEEDRETFMNKFIYEIARRHPFLYAPTILLWAARYDKIIPSCCKAENAVECFQTKAATVTKELRESSLLNQHACAVMKNFGTRTFQAITVTKLSQKFTKVNFTEIQKLVLDVAHVHEHCCRGDVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQESQALAKRSCGLFQKLGEYYLQNAFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQTMKQEFLINLVKQKPQITEEQLEAVIADFSGLLEKCCQGQEQEVCFAEEGQKLISKTRAALGV

SEQ ID NO: 7 - protein, 449 aa, Homo sapiens
  CHAIN (1)..(449): alpha-tubulin <s300> NCBI/AAA21350.1  GI161729 1994-09-07 (1)..(449)
MREVISIHVGQGGIQVGNACWELFCLEHGIQPDGQMPSDKTIGGGDDAFNTFFSETGAGKHVPRAVFLDLEPTVIDEVRTGTYRQLFHPEQLISGKEDAANNFARGHYTIGKEIVDLCLDRIRKLADNCTGLQGFLVFNSVGGGTGSGLGSLLLERLSVDYGKKSKLGFTIYPSPQVSTAVVEPYNSILSTHSLLEHTDVAVMLDNEAIYDICRRNLDIERPTYTNLNRLIAQVISSLTASLRFDGALNVDITEFQTNLVPYPRIHFMLSSYAPIISAEKAYHEQLSVAEITNSAFEPANMMAKCDPRHGKYMACSMMYRGDVVPKDVNASIATIKTKRTIQFVDWCPTGFKVGINYQPPTVVPGGDLAKVMRAVCMISNSTAIAEVFSRLDHKFDLMYAKRAFVHWYVGEGMEEGEFSEAREDLAALEKDYEEVGIETAEGEGEEEGY

SEQ ID NO: 8 - protein, 463 aa, Homo sapiens
  CHAIN (1)..(463): transformation upregulated nuclear protein (hnRNP K) <s300> NCBI/P61978  GI48429103 2006-01-24 (1)..(463)
METEQPEETFPNTETNGEFGKRPAEDMEEEQAFKRSRNTDEMVELRILLQSKNAGAVIGKGGKNIKALRTDYNASVSVPDSSGPERILSISADIETIGEILKKIIPTLEEGLQLPSPTATSQLPLESDAVECLNYQHYKGSDFDCELRLLIHQSLAGGIIGVKGAKIKELRENTQTTIKLFQECCPHSTDRVVLIGGKPDRVVECIKIILDLISESPIKGRAQPYDPNFYDETYDYGGFTMMFDDRRGRPVGFPMRGRGGFDRMPPGRGGRPMPPSRRDYDDMSPRRGPPPPPPGRGGRGGSRARNLPLPPPPPPRGGDLMAYDRRGRPGDRYDGMVGFSADETWDSAIDTWSPSEWQMAYEPQGGSGYDYSYAGGRGSYGDLGGPIITTQVTIPKDLAGSIIGKGGQRIKQIRHESGASIKIDEPLEGSEDRIITITGTQDQIQNAQYLLQNSVKQYSGKFF

SEQ ID NO: 9 - protein, 151 aa, Homo sapiens
  CHAIN (1)..(151): chain A, nucleoside triphosphate <s300> NCBI/1NUE_A  GI1421609 1998-09-04 (1)..(151)
ANLERTFIAIKPDGVQRGLVGEIIKRFEQKGFRLVAMKFLRASEEHLKQHYIDLKDRPFFPGLVKYMNSGPVVAMVWEGLNVVKTGRVMLGETNPADSKPGTIRGDFCIQVGRNIIHGSDSVKSAEKEISLWFKPEELVDYKSCAHDWVYE

SEQ ID NO: 10 - protein, 279 aa, Homo sapiens
  CHAIN (1)..(279): cytokeratin 8 <s300> NCBI/CAA31376.1  GI30313 2005-04-18 (1)..(279)
MNKVELESRLEGLTDEINFLRQLYEEEIRELQSQISDTSVVLSMDNSRSLDMDSIIAEVKAQYEDIANRSRAEAESMYQIKYEELQSLAGKHGDDLRRTKTEISEINRNISRLQAEIEGLKGQRASLEAAIADAEQRGELAIKDANAKLSELEAALQRAKQDMARQLREYQELMNVKLALDIEIATYRKLLEGEESRLESGMQNMSIHTKTTGGYAGGLSSAYGDLTDPGLSYSLGSSFGSGAGSSSFSRTSSSRAVVVKKIETRDGKLVSESSDVLPK

SEQ ID NO: 11 - protein, 355 aa, Homo sapiens
  CHAIN (1)..(355): heterogeneous nuclear ribonucleoprotein D0 (hnRNP D0) <s300> NCBI/Q14103  GI13124489 2006-01-24 (1)..(355)
MSEEQFGGDGAAAAATAAVGGSAGEQEGAMVAATQGAAAAAGSGAGTGGGTASGGTEGGSAESEGAKIDASKNEEDEGHSNSSPRHSEAATAQREEWKMFIGGLSWDTTKKDLKDYFSKFGEVVDCTLKLDPITGRSRGFGFVLFKESESVDKVMDQKEHKLNGKVIDPKRAKAMKTKEPVKKIFVGGLSPDTPEEKIREYFGGFGEVESIELPMDNKTNKRRGFCFITFKEEEPVKKIMEKKYHNVGLSKCEIKVAMSKEQYQQQQQWGSRGGFAGRARGRGGGPSQNWNQGYSNYWNQGYGNYGYNSQGYGGYGGYDYTGYNNYYGYGDYSNQQSGYGKVSRRGGHQNSYKPY

SEQ ID NO: 12 - protein, 290 aa, Homo sapiens
  CHAIN (1)..(290): heterogeneous nuclear ribonucleoprotein C isoform b <s300> NCBI/NP_004491.1  GI4758544 2005-12-11 (1)..(290)
MASNVTNKTDPRSMNSRVFIGNLNTLVVKKSDVEAIFSKYGKIVGCSVHKGFAFVQYVNERNARAAVAGEDGRMIAGQVLDINLAAEPKVNRGKAGVKRSAAEMYGSSFDLDYDFQRDYYDRMYSYPARVPPPPPIARAVVPSKRQRVSGNTSRRGKSGFNSKSGQRGSSKSGKLKGDDLQAIKKELTQIKQKVDSLLENLEKMEKEQSKQAVEMKNDKSEEEQSSSSVKKDETNVKMESEGGADDSAEEGDLLDDDDNEDRGDDQLELIKDDEKEAEEGEDDRDSANGG

SEQ ID NO: 13 - protein, 372 aa, Homo sapiens
  CHAIN (1)..(372): heterogeneous nuclear ribonucleoprotein A1 isoform b <s300> NCBI/NP_112420.1  GI14043070 2005-12-14 (1)..(372)
MSKSESPKEPEQLRKLFIGGLSFETTDESLRSHFEQWGTLTDCVVMRDPNTKRSRGFGFVTYATVEEVDAAMNARPHKVDGRVVEPKRAVSREDSQRPGAHLTVKKIFVGGIKEDTEEHHLRDYFEQYGKIEVIEIMTDRGSGKKRGFAFVTFDDHDSVDKIVIQKYHTVNGHNCEVRKALSKQEMASASSSQRGRSGSGNFGGGRGGGFGGNDNFGRGGNFSGRGGFGGSRGGGGYGGSGDGYNGFGNDGGYGGGGPGYSGGSRGYGSGGQGYGNQGSGYGGSGSYDSYNNGGGGGFGGGSGSNFGGGGSYNDFGNYNNQSSNFGPMKGGNFGGRSSGPYGGGGQYFAKPRNQGGYGGSSSSSSYGSGRRF

SEQ ID NO: 14 - protein, 679 aa, Homo sapiens
  CHAIN (1)..(679): MTHSP75 <s300> NCBI/AAA67526.1  GI292059 1995-05-26 (1)..(679)
MISASRAAAARLVGAAASRGPTAARHQDSWNGLSHEAFRLVSRRDYASEAIKGAVVGIDLGTTNSCVAVMEGKQAKVLENAEGARTTPSVVAFTADGERLVGMPAKRQAVTNPNNTFYATKRLIGRRYDDPEVQKDIKNVPFKIVRASNGDAWVEAHGKLYSPSQIGAFVLMKMKETAENYLGHTAKNAVITVPAYFNDSQRQATKDAGQISGLNVLRVINEPTAAALAYGLDKSEDKVIAVYDLGGGTFDISILEIQKGVFEVKSTNGDTFLGGEDFDQALLRHIVKEFKRETGVDLTKDNMALQRVREAAEKAKCELSSSVQTDINLPYLTMDSSGPKHLNMKLTRAQFEGIVTDLIRRTIAPCQKAMQDAEVSKSDIGEVILVGGMTRMPKVQQTVQDLFGRAPSKAVNPDEAVAIGAAIQGGVLAGDVTDVLLLDVTPLSLGIETLGGVFTKLINRNTTIPTKKSQVFSTAADGQTQVEIKVCQGEREMAGDNKLLGQFTLIGIPPAPRGVPQIEVTFDIDANGIVHVSAKDKGTRREQQIVIQSSGGLSKDDIENMVKNAEKYAEEDRRKKERVEAVNMAEGIIHDTETKMEEFKDQLPADECNKLKEEISKMRELLARKDSETGENIRQAASSLQQASLKLFEMAYKKMASEREGSGSSGTGEQKEDQKEEKQ

SEQ ID NO: 15 - protein, 216 aa, Homo sapiens [sequence omitted]

SEQ ID NO: 16 - protein, 115 aa, Homo sapiens
  CHAIN (1)..(115): ribosomal protein P2; 60S acidic ribosomal protein P2 <s300> NCBI/NP_000995.1  GI4506671 2005-10-28 (1)..(115)
MRYVASYLLAALGGNSSPSAKDIKKILDSVGIEADDDRLNKVISELNGKNIEDVIAQGIGKLASVPAGGAVAVSAAPGSAAPAAGSAPAAAEEKKDEKKEESEESDDDMGFGLFD

SEQ ID NO: 17 - protein, 69 aa, Homo sapiens [sequence omitted]

SEQ ID NO: 18 - protein, 278 aa, Homo sapiens
  CHAIN (1)..(278): human pre-mRNA splicing factor SF2p32, complete sequence <s300> NCBI/AAA73055.1  GI338043 1993-08-03 (1)..(278)
LRCVPRVLGSSVAGLRAAAPASPFRQLLQPAPRLCTRPFGLLSVRAGSERRPGLLRPRGPCACGCGCGSLHTDGDKAFVDFLSDEIKEERKIQKHKTLPKMSGGWELELNGTEAKLVRKVAGEKITVTFNINNSIPPTFDGEEEPSQGQKVEEQEPELTSTPNFVVEVIKNDDGKKALVLDCHYPEDEVGQEDEAESDIFSIREVSFQSTGESEWKDTNYTLNTDSLDWALYDHLMDFLADRGVDNTFADELVELSTALEHQEYITFLEDLKSFVKSQ